(19) United States
(12) Patent Application Publication
(10) Pub. No.: US 2014/0080768 A1
(43) Pub. Date: Mar. 20, 2014

(54) PRODRUGS OF DHODH INHIBITORS AND THEIR USES

(71) Applicants: Alan Palmer, Maidenhead (GB); Michael J. Hudson, Great Missenden (GB); Richard Todd, Burnham (GB); Patrick Camilleri, Stevenage (GB)

(72) Inventors: Alan Palmer, Maidenhead (GB); Michael J. Hudson, Great Missenden (GB); Richard Todd, Burnham (GB); Patrick Camilleri, Stevenage (GB)

(21) Appl. No.: 13/622,606

(22) Filed: Sep. 19, 2012

(51) Int. Cl. C07D 213/74; C07D 405/12

(52) U.S. Cl. CPC C07D 213/74; C07D 405/12
USPC 514/23; 546/312; 514/352; 544/131; 514/235.5; 544/360; 514/253.01; 546/283.7; 514/336; 536/55; 546/263; 548/309.4

(57) ABSTRACT

The invention generally relates to Pro-Drugs of dihydroorotate dehydrogenase (DHODH) inhibitors and methods of use thereof. In certain embodiments, the invention provides a DHODH inhibitor compound including a cleavable functional group that increases bioavailability as compared to a form of the DHODH inhibitor without the functional group, rendering the former more suitable for therapeutic use.

### FIELD OF THE INVENTION

The invention generally relates to prodrugs of dihydroorotate dehydrogenase (DHODH) inhibitors and methods of use thereof.

### BACKGROUND

When cells proliferate, they require a supply of nucleotides for expressing and copying the genetic material as well as for a variety of other metabolic processes. Those nucleotides can be supplied by de novo nucleotide synthesis pathways. One important step in the de novo synthesis pathway of pyrimidine nucleotides is the oxidation of dihydroorotate to form orotate. That reaction is catalyzed by dihydroorotate dehydrogenase (DHODH) and that step is one of the rate-limiting steps in the pyrimidine nucleotide synthesis pathway. Under normal circumstances the intracellular pool of pyrimidine nucleotides can be replenished by a salvage pathway in which pyrimidine nucleotides are recycled. Whilst this DHODH-independent mechanism is sufficient for resting lymphocytes, ‘activated’ and proliferating lymphocytes need to substantially increase the available pyrimidine and so become dependent on de novo pyrimidine synthesis.

The DHODH enzyme sits on the mitochondrial membrane and uses cytochrome C in the electron transport chain as an electron acceptor for the oxidation of dihydroorotate to orotate. Since orotate is a necessary intermediate in pyrimidine nucleotide synthesis, and since pyrimidine nucleotides are required for DNA replication, gene expression, and carbohydrate metabolism, inhibition of the DHODH enzyme can inhibit cell growth.

Further, rapidly proliferating cells require pyrimidines not only for cellular growth, but also for protein glycosylation, membrane lipid biosynthesis and strand break repair. See Fairbanks, et al., J. Biol. Chem. 270:29682-29689 (1995). To meet that increased demand, substantial quantities of pyrimidine nucleotides must be produced in rapidly proliferating cells. For that reason, DHODH inhibitors are attractive candidates for treating proliferative disorders. See Liu, S., et al., Structure 8:25-31 (2000). In fact, studies have shown that DHODH inhibitors can stop the proliferation of tumor cells in some circumstances. See Loffler, On the role of dihydroorotate dehydrogenase in growth cessation of Ehrlich Ascites tumor cells cultured under oxygen deficiency, Eur. J. Biochem. 107:207-215 (1980).

Some tissues (e.g. cells of the hematopoietic system and the gastrointestinal lining) lack the ability to perform de novo pyrimidine synthesis and so are largely unaffected by the antiproliferative effects of DHODH inhibitors, reducing the risk of side effects such as a reduced number of blood cells (cytopenia). Some animals, such as protozoan Apicomplexan parasites of the Plasmodium Species, also lack pyrimidine salvage enzymes and in these cases the de novo pathway regulated by DHODH provides the only source of pyrimidines for cell growth. Thus, DHODH inhibitors can also be expected to provide therapeutic benefit in the treatment of infectious disorders where the infectious agent has little or no pyrimidine salvage enzymes. See, e.g.: Phillips and Rathod, Infect Disord Drug Targets 10:226-239 (2010).

Other circumstances in which DHODH inhibitors have been identified as candidates for the clinical control of rapid cell division include activated immune cells, diseased skin cells, cancers, and infectious agents. Examples of DHODH inhibitors used or being developed for proliferative disorders include brequinar, leflunomide, and teriflunomide. Inhibitors of DHODH have further been disclosed for the treatment or prevention of autoimmune diseases, immune and inflammatory diseases, angioplastic-related disorders, viral, bacterial, and protozoic diseases.

A problem commonly associated with known compounds used to inhibit DHODH is that those compounds have poor bioavailability. See, e.g., U.S. Pub. 2012/0035175 to Ammendola, et al., and U.S. Pub. 2012/0003183 to Gonzalez, et al. After oral administration, it is thought that such DHODH inhibitors show poor resorption from the intestines into circulation, which may be related to poor pharmacokinetic properties as a consequence of, for example, poor solubility or lipophilicity. Since only substances with appropriate pharmacokinetic properties will be resorbed, compounds with poor pharmacokinetic properties exhibit poor uptake from the gastrointestinal tract and have limited pharmaceutical efficacy. For example, some categories of DHODH inhibitors are known to have aqueous solubilities as low as 1 mg/ml, which effectively prevents their resorption from the gastrointestinal (GI) tract.

Since the poor bioavailability of many DHODH inhibitors limits their uptake from the GI tract, oral administration of those compounds may result in plasma levels of the drug that are insufficient to inhibit the production of orotate, with the result that these otherwise promising compounds are not pharmaceutically useful. Thus, while drugs based on DHODH inhibitors have shown potential for the treatment of a number of diseases, existing formulations are inadequate for the treatment of those diseases on a routine basis.

### SUMMARY

The invention generally provides dihydroorotate dehydrogenase (DHODH) inhibitor compounds that are able to survive the ‘first-pass’ metabolism associated with oral administration and have improved resorption from the intestines into circulation compared to known DHODH inhibitors. In this manner, compounds of the invention also have improved bioavailability when compared to known DHODH inhibitors and are able to provide beneficial pharmaceutical properties for treating autoimmune, immune, inflammatory, proliferative and other disorders. To avoid first-pass metabolism and achieve improved bioavailability, compounds of the invention are formulated with a cleavable functional group that improves pharmacokinetic properties by, for example, increasing solubility, masking hydrogen bonding groups, or introducing lipophilicity. Such compounds are described herein as ‘Pro-Drugs’. Accordingly, compounds of the invention exhibit good uptake from the GI tract resulting in improved plasma levels relative to the plasma levels obtained by administration of non-Pro-Drug formulations of the same DHODH inhibitors.

Compositions of the invention include any compound that results in an active DHODH inhibitor being produced within the body upon cleavage of the associated functional group that improves bioavailability. DHODH inhibitors include, for example, DHODH inhibitors known in the art such as those disclosed in U.S. Pub. 2012/0003183 and WO 2010/115736. In certain embodiments, the functional group includes, for example, an amino acid, a carbamate, a phosphate, a phosphonate, a sulphamic acid, an ester, an amide, a heterocycloalkyl, a carbonate ester, ethyl morpholine, ethyl piperazine, dimethyl-dioxyl-2-one, ethyl dimethylpropanoate, 6-(hydroxymethyl)tetrahydropyran-3,4,5-triol, propanediyl diacetate, or an azo derivative. The functional group is attached, for example, to a carboxylic acid of the DHODH inhibitor as an ester, carbonate, amide, carbamate, ether, phosphate, oxime, or imine.

In certain aspects, the invention provides a pro-Drug of a DHODH inhibitor compound or a pharmaceutically acceptable salt thereof, in which the compound or the salt thereof includes a functional group that increases bioavailability as compared to a form without the functional group and in which the compound is represented by formula (I), (II), or (III):

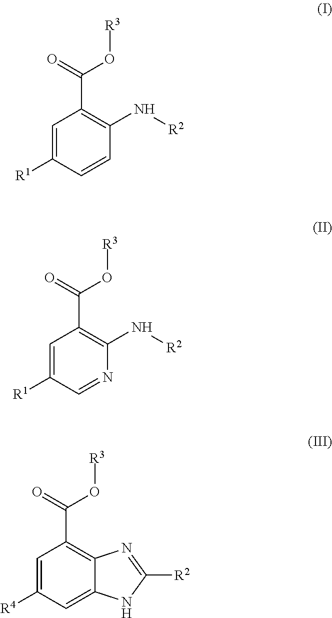

in which R¹ is H or an alkyl group; R² is a biaryl or a heterocyclic biaryl, and the biaryl or the heterocyclic biaryl is optionally substituted with one or more halogen and optionally substituted with one or more CF₃; R³ is selected from an amino acid, a carbamate, a phosphate, a phosphonate, a sulphamic acid, an ester, an amide, a heterocycloalkyl, dimethyl-dioxyl-2-one, ethyl-dimethylpropanoate, 6-hydroxymethyl-tetrahydropyran-2,3,5-triol, propanediyl-diacetate, and an azo derivative; and R⁴ is a halogen or CH₃. In some embodiments, R³ is one of:

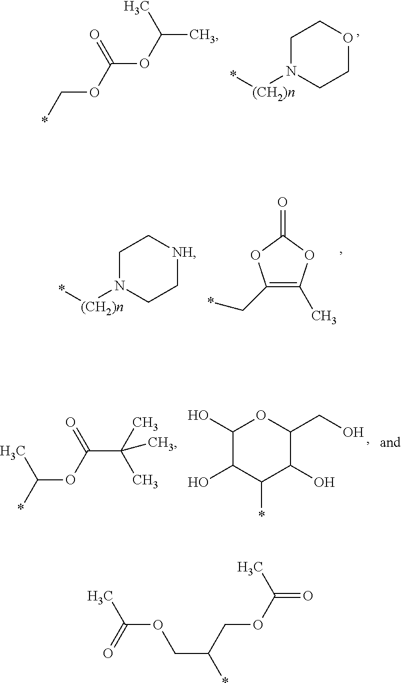

where n is 1, 2, 3, or 4 and * represents the point of attachment.

In certain embodiments, n is 2 and R³ is

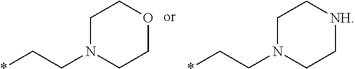

In certain embodiments, the DHODH inhibitor compound is represented by formula (IV):

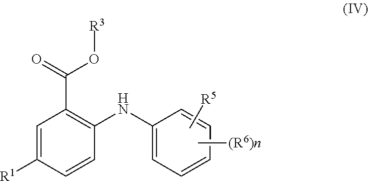

wherein R⁵ is an aryl or a heterocyclic aryl, wherein the aryl or the heterocyclic aryl is optionally substituted with one or more halogen and optionally substituted with one or more CF₃, and
R⁶ is a halogen or CF₃ and n=0, 1, 2, 3, or 4,
or a pharmaceutically acceptable salt thereof.

In certain embodiments, the DHODH inhibitor compound is represented by formula (IV) and R³ is one of:

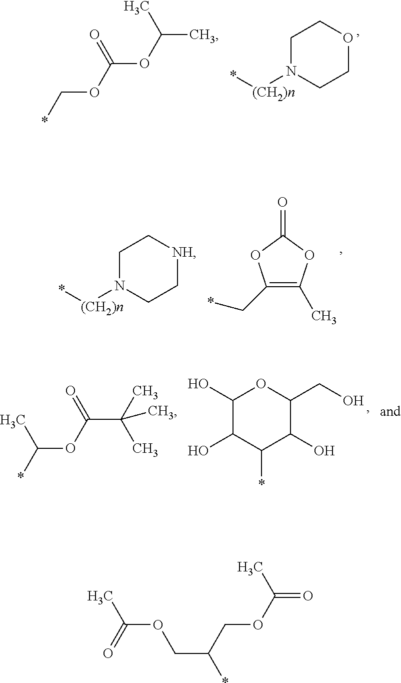

where n is 1, 2, 3, or 4 and * represents the point of attachment.
In certain embodiments, R³ is

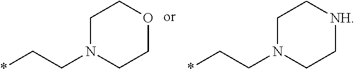

In some embodiments, the DHODH inhibitor compound is represented by formula (V):

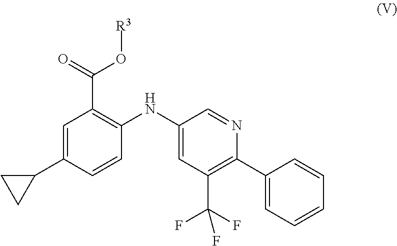

or a pharmaceutically acceptable salt thereof.

In certain embodiments, the DHODH inhibitor compound is represented by formula (V) and R³ is one of:

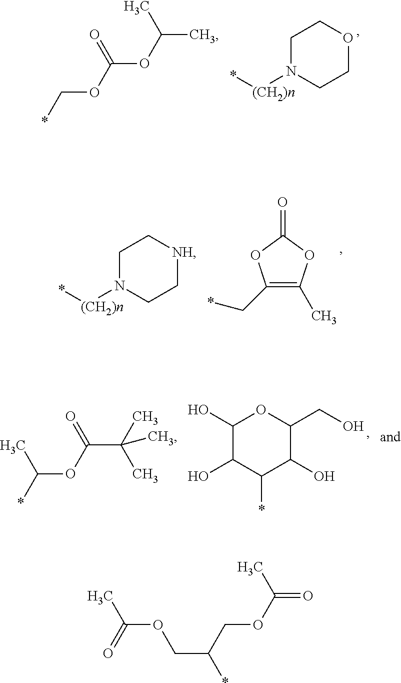

where n is 1, 2, 3, or 4 and * represents the point of attachment.
In certain embodiments, R³ is

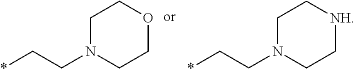

In some embodiments, the DHODH in inhibitor compound is represented by formula (VI):

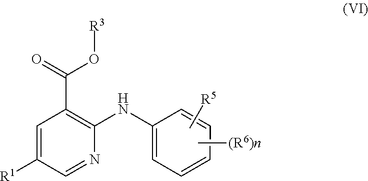

wherein R⁵ is an aryl or a heterocyclic aryl, wherein the aryl or the heterocyclic aryl is optionally substituted with one or more halogen and optionally substituted with one or more CF₃, and
R⁶ is a halogen or CF₃ and n=0, 1, 2, 3, or 4,
or a pharmaceutically acceptable salt thereof.

In certain embodiments, the DHODH inhibitor compound is represented by formula (VI) and R³ is one of:

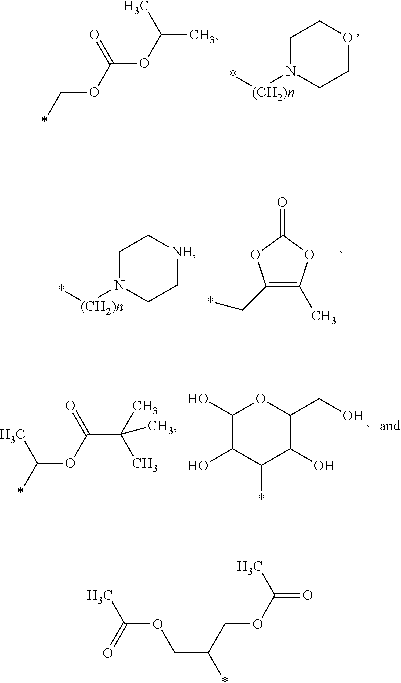

where n is 1, 2, 3, or 4 and * represents the point of attachment.
In certain embodiments, R³ is

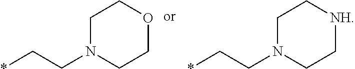

In some embodiments, the DHODH in inhibitor compound is represented by formula (VII):

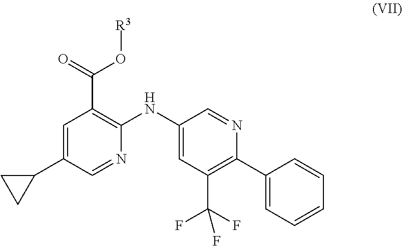

or a pharmaceutically acceptable salt thereof.

In certain embodiments, the DHODH inhibitor compound is represented by formula (VII) and R³ is one of:

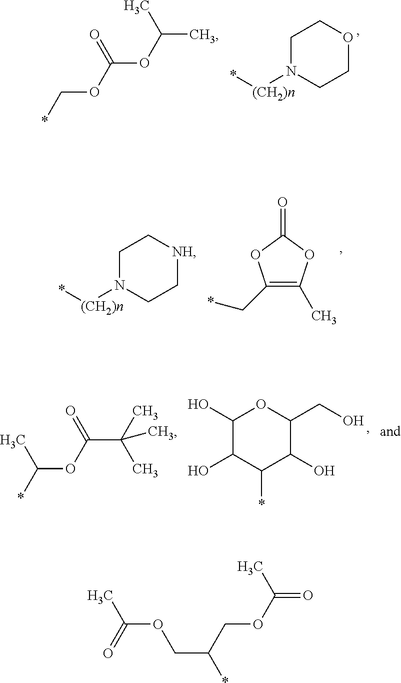

where n is 1, 2, 3, or 4 and * represents the point of attachment.
In certain embodiments, R³ is

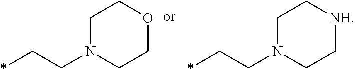

In some embodiments, the DHODH inhibitor compound is represented by formula (VIII):

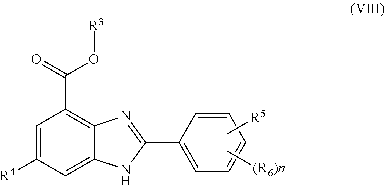

wherein R⁵ is an aryl or a heterocyclic aryl, wherein the aryl or the heterocyclic aryl is optionally substituted with one or more halogen and optionally substituted with one or more CF₃, and
R⁶ is a halogen or CF₃ and n=0, 1, 2, 3, or 4,
or a pharmaceutically acceptable salt thereof.

In certain embodiments, the DHODH inhibitor compound is represented by formula (VIII) and R³ is selected from the group consisting of:

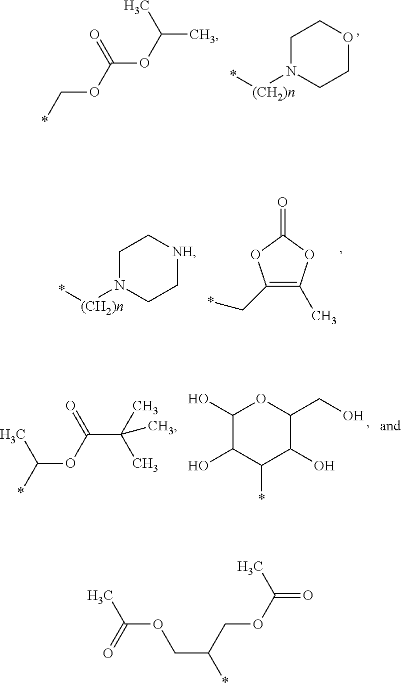

where n is 1, 2, 3, or 4 and * represents the point of attachment.
In certain embodiments, R³ is

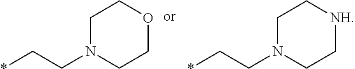

In some embodiments, the DHODH inhibitor compound is represented by formula (IX):

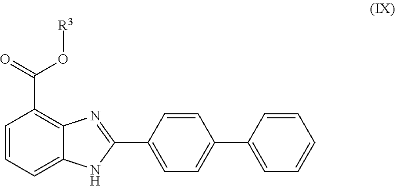

in which the biphenyl is optionally substituted with one or two halogens, or a pharmaceutically acceptable salt thereof.

In certain embodiments, the DHODH inhibitor compound is represented by formula (IX) and R³ is one of:

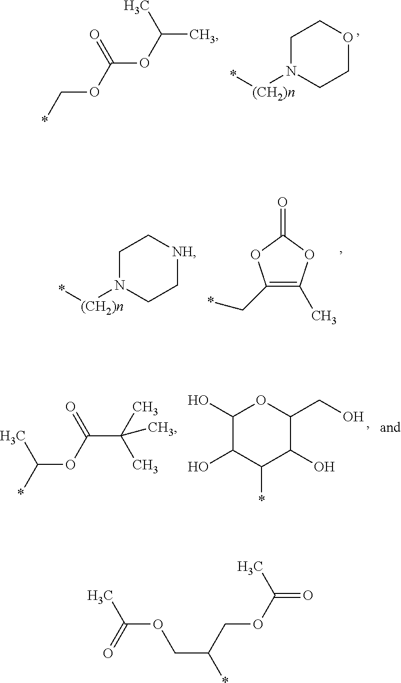

where n is 1, 2, 3, or 4 and * represents the point of attachment.
In certain embodiments, R³ is

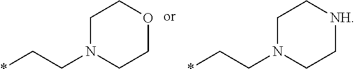

In certain embodiments, the compound is:

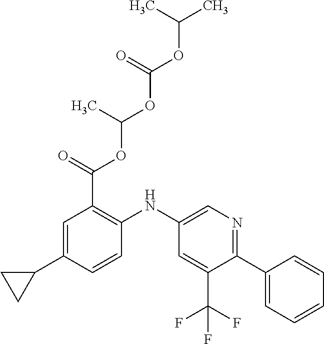 [SMILES: CC(C)OC(=O)OC(C)OC(=O)c1cc(C2CC2)ccc1Nc1cnc(-c2ccccc2)c(C(F)(F)F)c1]

or a pharmaceutically acceptable salt thereof.

In certain embodiments, the compound is:

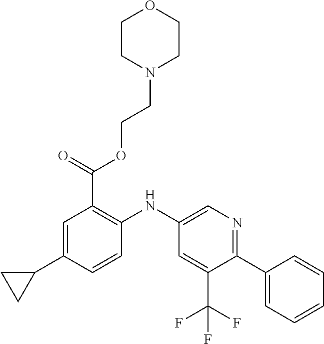 [SMILES: O=C(OCCN1CCOCC1)c1cc(C2CC2)ccc1Nc1cnc(-c2ccccc2)c(C(F)(F)F)c1]

or a pharmaceutically acceptable salt thereof.

In certain embodiments, the compound is:

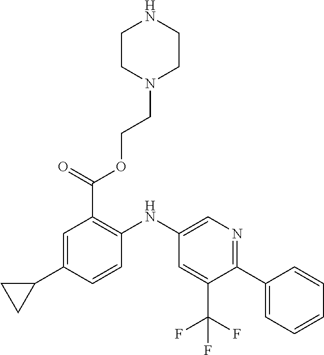 [SMILES: O=C(OCCN1CCNCC1)c1cc(C2CC2)ccc1Nc1cnc(-c2ccccc2)c(C(F)(F)F)c1]

or a pharmaceutically acceptable salt thereof.

In certain embodiments, the compound is:

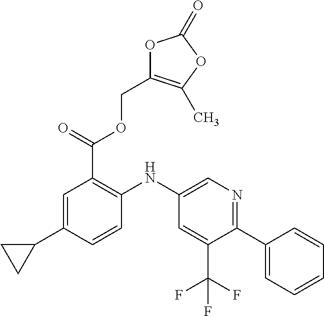 [SMILES: Cc1oc(=O)oc1COC(=O)c1cc(C2CC2)ccc1Nc1cnc(-c2ccccc2)c(C(F)(F)F)c1]

or a pharmaceutically acceptable salt thereof.

In certain embodiments, the compound is:

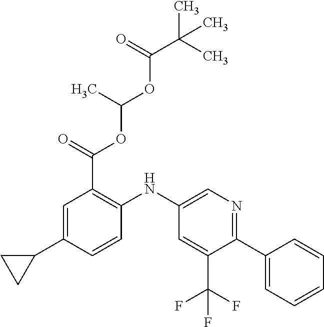 [SMILES: CC(OC(=O)c1cc(C2CC2)ccc1Nc1cnc(-c2ccccc2)c(C(F)(F)F)c1)OC(=O)C(C)(C)C]

or a pharmaceutically acceptable salt thereof.

In certain embodiments, the compound is:

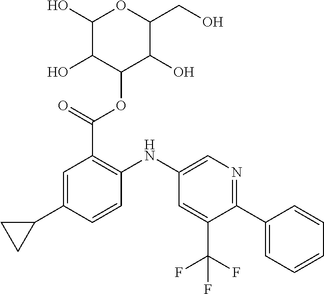 [SMILES: O=C(OC1C(O)C(O)OC(CO)C1O)c1cc(C2CC2)ccc1Nc1cnc(-c2ccccc2)c(C(F)(F)F)c1]

or a pharmaceutically acceptable salt thereof.

In certain embodiments, the compound is:

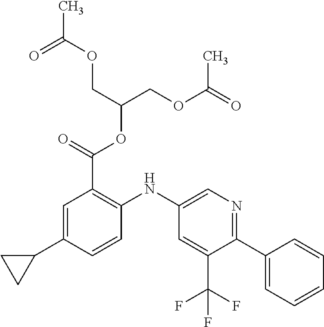 [SMILES: CC(=O)OCC(COC(C)=O)OC(=O)c1cc(C2CC2)ccc1Nc1cnc(-c2ccccc2)c(C(F)(F)F)c1]

or a pharmaceutically acceptable salt thereof.

In certain aspects, the invention provides a method of treating a condition or disease that is susceptible to amelioration by inhibition of DHODH, the method involving administering to a subject a therapeutically effective dose of a DHODH inhibitor as described above, or a pharmaceutically acceptable salt thereof, in which the compound or the salt thereof includes a functional group that increases bioavailability as compared to a form without the functional group.

### DETAILED DESCRIPTION

The invention provides compounds that are Pro-Drugs of inhibitors of the enzyme dihydroorotate dehydrogenase (DHODH) and methods of use thereof for the treatment of autoimmune conditions, inflammatory diseases and proliferative disorders. Compounds of the invention include a DHODH inhibitor linked (e.g., covalently) to a functional group that improves the bioavailability of the DHODH inhibitor as compared to a form of the compound without the functional group.

Any suitable DHODH inhibitor may be used in a Pro-Drug of the invention. In certain embodiments, the DHODH inhibitor has the following general structures represented by formulas (I), (II), and (III):

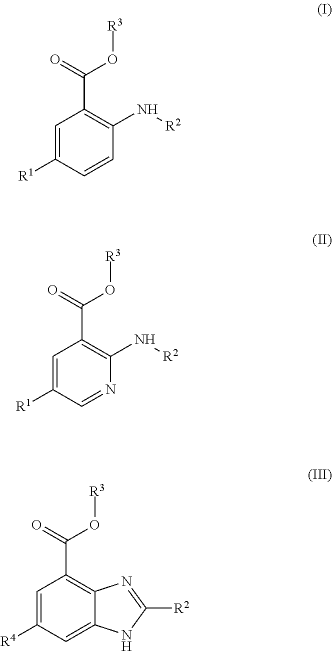

in which R¹ is H or an alkyl group; R² is a biaryl or a heterocyclic biaryl, and the biaryl or the heterocyclic biaryl is optionally substituted with one or more halogen and optionally substituted with one or more CF₃; R³ is selected from an amino acid, a carbamate, a phosphate, a phosphonate, a sulphamic acid, an ester, an amide, a heterocycloalkyl, dimethyl-dioxyl-2-one, ethyl-dimethylpropanoate, 6-hydroxymethyl-tetrahydropyran-2,3,5-triol, propanediyl-diacetate, and an azo derivative; and R⁴ is a halogen or CH₃.

In some embodiments, R³ is one of:

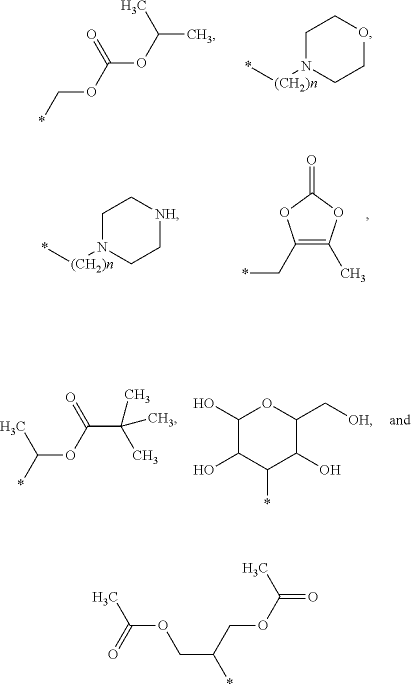

where n is 1, 2, 3, or 4 and * represents the point of attachment.

In certain embodiments, R³ is

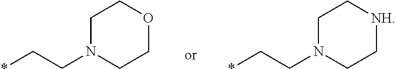 [SMILES: *CCN1CCOCC1 ; *CCN1CCNCC1]

Examples of DHODH inhibitors for use in Pro-Drugs of the invention include brequinar, leflunomide and teriflunomide. Brequinar is an anticancer drug candidate. See, e.g., Chen S F, et al., Cancer Res. 52:3521-3527 (1992). Leflunomide, sold under the trade name ARAVA by Sanofi-Aventis, was approved by the United States Food and Drug Administration (FDA) for the treatment of rheumatoid arthritis in 1998 and by the European Medicines Agency for the treatment of psoriatic arthritis in 2004. (EP 0 780 128, WO 97/34600) (Pinto P, Dougados, M. 2006 Acta Reumatol Port. 31: 215-224; Herrmann, M. L., et al., 2000 Immunopharmacology 47: 273-289). Upon administration, leflunomide is metabolized to teriflunomide, which inhibits the DHODH enzyme. Emerging evidence supports the use of leflunomide in organ transplantation and as an antiviral agent (e.g. Teschner S, Burst V., 2010 Immunotherapy. 2:637-650; Wu, J K, Harris MT., 2008 Ann Pharmacotherapy. 42:1679-1685). Teriflunomide has entered Phase III clinical trials for multiple sclerosis (Palmer A M 2010, Curr Opin Investig Drugs 11:1313-1323).

Further examples of active DHODH inhibitor compounds with potential in Pro-Drugs of the invention include, for example, those disclosed in WO 06/044741 and U.S. Pub. 2007/0197643; WO/2006/022442; WO/2006/001961 and U.S. Pub. 2007/0219224; WO/2004/056747; WO/2004/056746; WO 03/006425, U.S. Pat. No. 7,423,057; WO 02/080897 and U.S. Pub. 2002/0177623; WO 99/45926; WO 2008/077635 and U.S. Pub. 2010/0145625; and WO 2009/021696 and U.S. Pub. 2011/0212945.

Compounds of the invention include a functional group that is cleavably linked to the DHODH inhibitor. The functional group improves the bioavailability of the DHODH inhibitor by modifying the solubility, stability, lipophilicity, hydrogen bonding, or other aspects of the pharmacokinetic properties of the active DHODH inhibitor. Pro-Drugs in general are discussed in Rautio, Prodrugs: design and clinical applications, Nature Reviews Drug Discovery 7:255-270 (2008).

Pro-Drug moieties for use in compounds of the invention include, for example, amino acids, carbamates, phosphates, phosphonates, sulphamic acids, esters, amides, heterocycloalkyls, carbonate esters, ethyl morpholine, ethyl piperazine, dimethyl-dioxyl-2-one, ethyl dimethylpropanoate, hydroxymethyl-tetrahydropyran triol, propanediyl diacetate, and azo derivatives. The Pro-Drug moiety is attached, for example, to a carboxylic acid of the DHODH inhibitor as an ester, carbonate, amide, carbamate, ether, phosphate, oxime, or imine. Pro-Drugs of amino-group bearing parent drugs are discussed in Simplicio, et al., Pro-Drugs for amines, Molecules 2008, 13, 519-547. Some examples of Pro-Drug moieties in the literature that have been used to modulate the solubility, stability and pharmacokinetic properties of active drug molecules bearing one or more amino groups are: natural amino acids, attached through the carboxyl group (e.g. Current Medicinal Chemistry, 2004, 11, 1241-1253; International Journal of Pharmaceutics 121 (1995) 157-167; WO 2005/046575 A2; J. Med. Chem. 2002, 45, 744; Eur. J. Med. Chem. 1991, 26, 143), carbamates (U.S. Pat. No. 7,060,259; U.S. Pat. No. 5,401,868; J. Med. Chem. 2004, 47, 2651), phosphates and phosphonate esters (J. Org. Chem. 1996, 61, 8636; Bioorg. and Med. Chem. Lett. 1998, 8, 3159), sulphamic acids (Bioorg & Med. Chem. Lett. 2001, 11 1093), esters (Pharmaceutical Research 1991, 8, 455), amides (J. Med. Chem. 2000, 43, 2530) and azo derivatives (Am. J. Gastroenterol. 2002, 97, 2939).

The present invention includes the realization that Pro-Drugs of DHODH inhibitors may improve pharmacokinetic properties of those DHODH inhibitors, relative to non-Pro-Drug formulations of those molecules, thereby improving and modulating plasma levels of the active drug. By judicious choice of the Pro-Drug moiety, it should be possible to tailor the exposure of the active compound in order to enhance bioavailability and optimize the pharmacokinetic-pharmacodynamic relationship in order to achieve the best possible risk-benefit profile.

Such Pro-Drugs may be useful in the therapy, whether prevention or treatment, of any of the disease or disorders discussed herein. Pro-Drugs of DHODH inhibitors have the potential to alter the pharmacokinetic properties of the parent molecule. The Pro-Drugs can be neutral, or contain a basic amino group. By altering the mode of ionization of the molecule in vivo, improvements in the bioavailability can often be obtained.

Embodiments of the invention include the following Pro-Drug moieties:

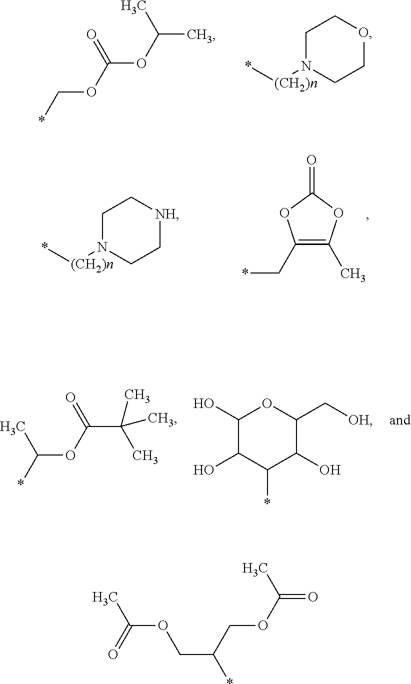

where n is 1, 2, 3, or 4 and * represents the point of attachment.

In certain embodiments, R³ is

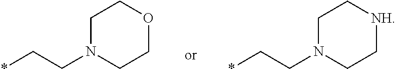 [SMILES: *CCN1CCOCC1 ; *CCN1CCNCC1]

Those Pro-Drug moieties may provide particularly beneficial bioavailability for DHODH inhibitors. In certain embodiments, those Pro-Drug moieties improve water solubility, improve lipophilicity, mask an otherwise exposed carbonate group, or a combination thereof, to improve bioavailability of the DHODH inhibitor.

The invention further provides pathways for the synthesis of Pro-Drugs. In general, the DHODH inhibitors according to formulae of this invention may be prepared from readily available starting materials or by standard synthetic techniques, such as those described in WO 2010/115736 or U.S. Pub. 2012/0028959, the contents of which are incorporated by reference in their entirety. Those techniques can be used to provide the starting point of Pathway A, below.

Pathway A illustrates the esterification of an acyl chloride of an active DHODH inhibitor with a Pro-Drug moiety in an alcohol form.

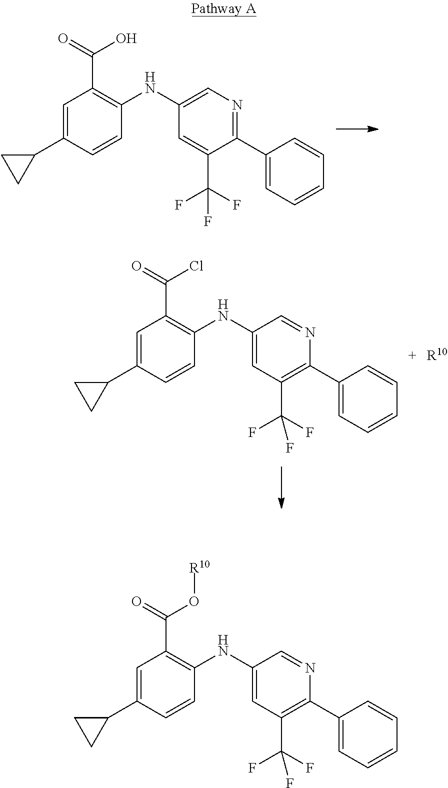

where R¹⁰ is one of

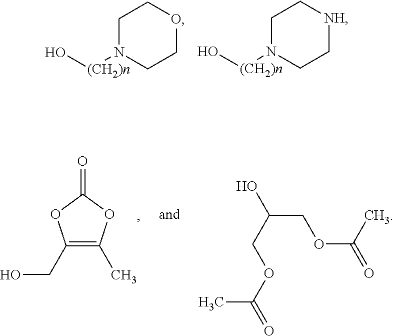

where n=1, 2, 3, or 4.

In some embodiments, 6-[(acetyloxy)methyl]-4-hydroxytetrahydro-2H-pyran-2,3,5-triyl triacetate is used in the synthesis via reaction and mild hydrolysis of a Pro-Drug in which the prodrug moiety is a 2,3,4-trihydroxy-6-(hydroxymethyl)tetrahydro-2H-pyran-4-yl group, as described in Pathway B.

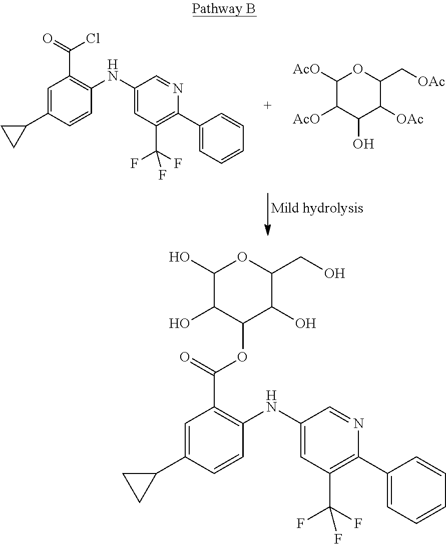 [SMILES: O=C(Cl)c1cc(C2CC2)ccc1Nc1cnc(-c2ccccc2)c(C(F)(F)F)c1 ; CC(=O)OCC1OC(OC(C)=O)C(OC(C)=O)C(O)C1OC(C)=O ; O=C(OC1C(O)C(O)OC(CO)C1O)c1cc(C2CC2)ccc1Nc1cnc(-c2ccccc2)c(C(F)(F)F)c1]

In certain embodiments, a carboxylic acid group on the active DHODH inhibitor is esterified with a Pro-Drug moiety according to Pathway C.

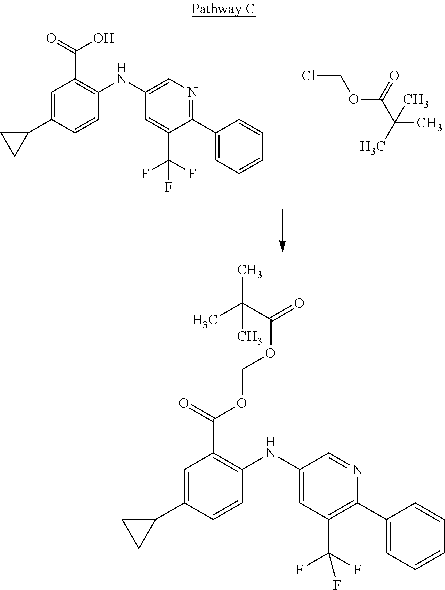

In some embodiments, prodrugs can be prepared exploiting an S_{N}2 reaction having the general form:

RX+R′CO₂→RCO₂R′+X

where R is the Pro-Drug moiety and R′CO₂ is the active DHODH inhibitor. For example, 1-chloroethyl propan-2-yl carbonate can be reacted with 5-cyclopropyl-2-{[6-phenyl-5-(trifluoromethyl)pyridin-3-yl]amino}benzoic acid according to the Pathway D, below, to synthesize a Pro-Drug of the invention.

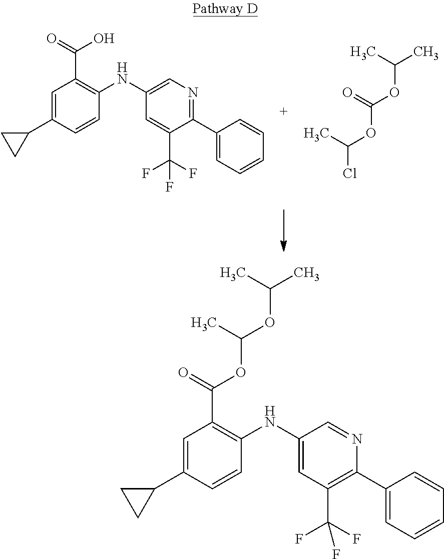

For Pro-Drugs containing a basic nitrogen, formulations according to the invention can include the free base as well as the hydrochloride, hydrobromide, sulphate, phosphate, toluenesulfonate, succinate, maleate, fumarate, or benzoate salts.

Pro-Drugs of the invention may be used to treat or prevent disorders including proliferative, immune, and autoimmune and inflammatory disorders, as well as any of the other disorders referenced herein. Further, in some embodiments, Pro-Drugs of the invention operate by exhibiting a differential effect on lymphocytes as compared to non-lymphoid cells.

Diseases or disorders in which DHODH inhibition plays a role include without limitation autoimmune disease, immune and inflammatory disease, cancer (including the treatment or prevention of metastasis), destructive bone disorders, traumatic injury, viral disease, bacterial disease and protozoic disease.

Autoimmune diseases which may be treated or prevented include without limit rheumatoid arthritis, psoriatic arthritis, multiple sclerosis, ankylosing spondylitis, acute disseminated encephalomyelitis, alopecia areata, antiphospholipid syndrome, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, celiac disease, cold agglutinin disease, diabetes mellitus type 1, eosinophilic fasciitis, gastrointestinal pemphigoid, Goodpasture's syndrome, Graves' disease, Guillain-Barré syndrome, Hashimoto's encephalopathy, Hashimoto's thyroiditis, idiopathic thrombocytopenic purpura, lupus erythematosus, Miller-Fisher syndrome, myasthenia gravis, pemphigus vulgaris, pernicious anemia, primary biliary cirrhosis, relapsing polychondritis, Sjögren's syndrome, temporal arteritis, transverse myelitis, ulcerative colitis, undifferentiated connective tissue disease, vasculitis, vitiligo, and Wegener's granulomatosis.

Immune and inflammatory diseases which may be prevented or treated with compounds of the invention include without limit asthma, rheumatoid arthritis, inflammatory bowel disease, diabetic cardiomyopathy, myocardial dysfunction, chronic obstructive pulmonary disease, respiratory disease syndrome, acute and chronic pancreatitis, graft versus host disease, chronic sarcoidosis, transplant rejection, contact dermatitis, atopic dermatitis, allergic rhinitis, conjunctivitis, Behcet syndrome, inflammatory eye conditions and uveitis. See, e.g., Leban, J., and Vitt, D., Human dihydroorotate dehydrogenase inhibitors, a novel approach for the treatment of autoimmune and inflammatory diseases, Arzneimittelforschung 61(1):66-72 (2011).

Bone disorders which may be prevented or treated include but are not limited to osteoporosis and acromegaly.

Cancers which may be prevented or treated include leukemia, oral cavity carcinomas, pulmonary cancers such as pulmonary adenocarcinoma, colorectal cancer, brain cancer bladder carcinoma, liver tumors, stomach tumors, liver tumors, colon tumors, prostate cancer, lung tumors, oral cavity carcinomas.

Viral diseases which may be prevented or treated include but are not limited to HIV infection, hepatitis and cytomegalovirus infection, herpes simplex virus infection, and meningitis.

Infectious diseases which may be prevented or treated include but are not limited to sepsis, septic shock, endotoxic shock, Gram negative sepsis, toxic shock syndrome, and protozoal diseases such as amoebic dysentery, trypanosomiasis, malaria and intestinal protozoal disease.

Further, DHODH has been validated as a target for the identification of new antimalarial chemotherapy and protozoic diseases. See, e.g., Phillips M A, Rathod, P K. 2010, Infect Disord Drug Targets 10(3):226-239 (2010).

Many organisms also have salvage pathways to recover purine and pyrimidine compounds obtained in the diet or released during nucleic acid turnover and degradation. While the ribose of nucleotides can be catabolized to generate energy, the nitrogenous bases do not serve as energy sources; their catabolism does not lead to products used by pathways of energy conservation. Due to the fact that DHODH inhibitors interfere with the de novo nucleotide synthesis pathway, cells that are able to advantageously exploit salvage pathways for pyrimidine nucleotides may be affected differentially by DHODH inhibitors than cells which require the production of abundant pyrimidine nucleotides via the de novo synthesis pathways.

In some embodiments, compounds of the invention prevent the expansion of activated and autoimmune lymphocytes by interfering with their cell cycle progression while non-lymphoid cells are able to use another pathway to make their ribonucleotides by use of salvage pyrimidine pathway, which makes them less dependent on de novo synthesis. See, e.g., Fox et al., Mechanism of action for leflunomide in rheumatoid arthritis, Clin Immunol 93(3):198-208 (1999).

Another realization is that DHODH inhibition may reduce the activity of core 2 β1,6-N-acetylglucosaminyltransferase (C2GnT). This enzyme defines a branch point in the O-linked glycan biosynthesis pathway that converts core 1 (i.e., Galb1,3GalNAca-O) to core 2 structures (i.e. Galb1,3[GlcNAc-β1,6]GalNAca-0. See, e.g., Beum and Cheng, Adv Exp Med Biol 491:279-312 (2001). The reaction requires uridine diphosphate N-acetylglucosamine (UDP-GlcNAc). Since DHODH inhibition reduces the concentration of uridine diphosphate (UDP), it may also lead to reduced C2GnT activity.

C2GnT plays a key role in the interaction between selectin molecules on the endothelium and counter-ligands on the leukocyte surface. For binding to occur, the counter ligand must be decorated with specific carbohydrate side-chains, such as 2,3-sialylated and a 1,3-fucosylated core 2 decorated O-glycans carrying the sialyl Lewis X (SLeX) motif as a capping group. See generally, Kansas, Selectins and their ligands: current concepts and controversies, Blood 88:3259-3287 (1996); and Yang, et al., The biology of P-selectin glycoprotein ligand-1: its role as a selectin counter receptor in leukocyte-endothelial and leukocyte-platelet interaction, Thromb Haemost 81(1):1-7 (1999). These side chains, which are produced by the activity of C2GnT, contribute to T-cell activation and inappropriate recruitment of leukocytes to the site of inflammation. Thus, reducing the activity of C2GnT may be responsible for the beneficial immunosuppressant and anti-proliferative effects of DHODH inhibition in human hyper-proliferative and inflammatory diseases.

Further, the relationship between C2GnT and DHODH inhibition may be exploited in developing dosing regimes of Pro-Drugs of the invention. The measurement of C2GnT activity or the concentration of the specific carbohydrate side-chains produced by C2GnT may therefore provide useful pharmacodynamic readout, alongside the measurement of DHODH activity, to permit optimization of the dosing regimen of DHODH inhibitors or their Pro-Drugs to help establish the best risk-benefit profile of such compounds.

The invention generally provides DHODH inhibitor Pro-Drugs and pharmaceutically effective compositions of DHODH inhibitor Pro-Drugs, as well as methods of use that include administering compounds or compositions of the invention for the treatment of a patient. Suitable routes of administration include oral, buccal, topical (including trans-dermal), etc. Each agent is preferably administered by the oral route.

The effective dosage of each agent can readily be determined by the skilled person, having regard to typical factors each as the age, weight, sex and clinical history of the patient.

A pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. No. 4,256,108, U.S. Pat. No. 4,166,452 and U.S. Pat. No. 4,265,874, to form osmotic therapeutic tablets for control release.

Formulations for oral use may also be presented as hard gelatin capsules in which the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

Formulations may also include complexes of the parent (unionized) compounds with derivatives of β-cyclodextrin, especially hydroxypropyl-β-cyclodextrin.

An alternative oral formulation, where control of GI tract hydrolysis of the prodrugs is sought, can be achieved using a controlled-release formulation, where the Pro-Drug is encapsulated in an enteric coating.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents such as a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan mono-oleate and condensation products of the said partial esters with ethylene oxide, for example polyoxyethylene sorbitan mono-oleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Each active agent may also be administered in the form of suppositories for rectal administration of the drug or Pro-Drug. These compositions can be prepared by mixing the drug or Pro-Drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions are suitable. Topical application includes the use of mouth washes and gargles.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A dihydroorotate dehydrogenase inhibitor compound comprising a cleavable functional group, the compound being represented by formula (I), (II), or (III): 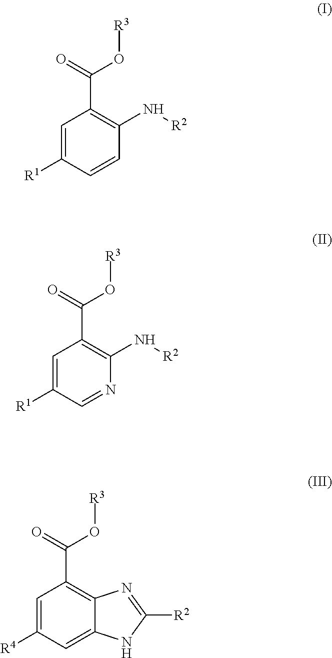
wherein R¹ is H or an alkyl group,
R² is a biaryl or a heterocyclic biaryl, and the biaryl or the heterocyclic biaryl is optionally substituted with one or more halogen and optionally substituted with one or more CF₃,
R³ is selected from an amino acid, a carbamate, a phosphate, a phosphonate, a sulphamic acid, an ester, an amide, a heterocycloalkyl, dimethyl-dioxyl-2-one, ethyl-dimethylpropanoate, 6-hydroxymethyl-tetrahydropyran-2,3,5-triol, propanediyl-diacetate, and an azo derivative; and
R⁴ is a halogen or CH₃.

2. The compound of claim 1, wherein R³ is selected from the group consisting of: 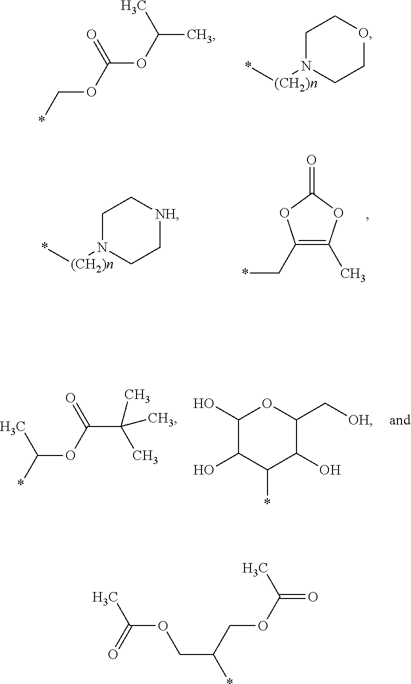
where n is 1, 2, 3, or 4 and * represents the point of attachment,
or a pharmaceutically acceptable salt thereof.

3. The compound of claim 2, wherein n is 2.

4. The compound of claim 1, represented by formula (IV): 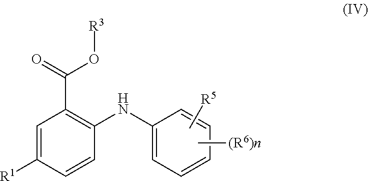
wherein R⁵ is an aryl or a heterocyclic aryl, wherein the aryl or the heterocyclic aryl is optionally substituted with one or more halogen and optionally substituted with one or more CF₃, and
R⁶ is a halogen or CF₃ and n=0, 1, 2, 3, or 4,
or a pharmaceutically acceptable salt thereof.

5. The compound of claim 4, wherein R³ is selected from the group consisting of: 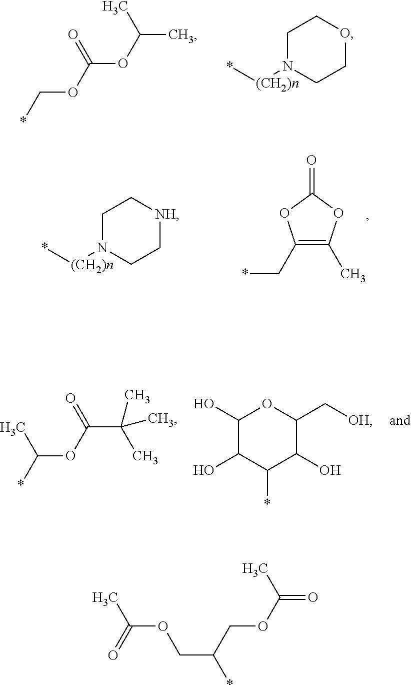
where n is 1, 2, 3, or 4 and * represents the point of attachment,
or a pharmaceutically acceptable salt thereof.

6. The compound of claim 1, represented by formula (V): 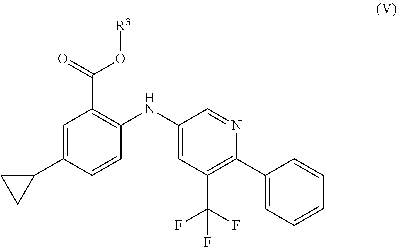
or a pharmaceutically acceptable salt thereof.

7. The compound of claim 6, wherein R³ is selected from the group consisting of: 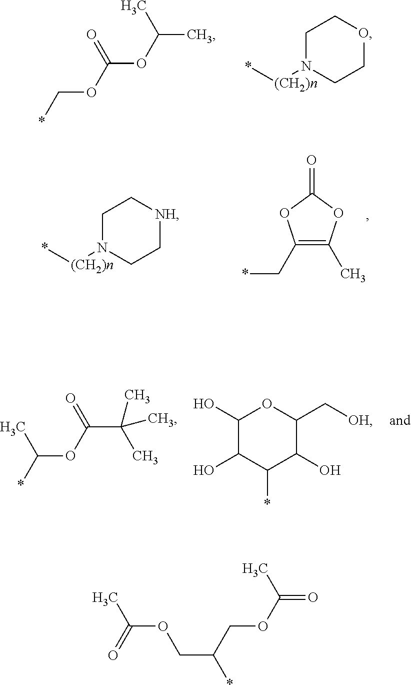
where n is 1, 2, 3, or 4 and * represents the point of attachment,
or a pharmaceutically acceptable salt thereof.

8. The compound of claim 1, represented by formula (V): 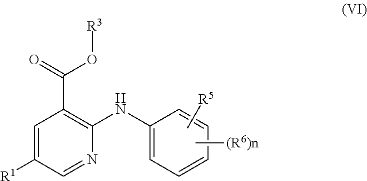
wherein R⁵ is an aryl or a heterocyclic aryl, wherein the aryl or the heterocyclic aryl is optionally substituted with one or more halogen and optionally substituted with one or more CF₃, and
R⁶ is a halogen or CF₃ and n=0, 1, 2, 3, or 4,
or a pharmaceutically acceptable salt thereof.

9. The compound of claim 8, wherein R³ is selected from the group consisting of: 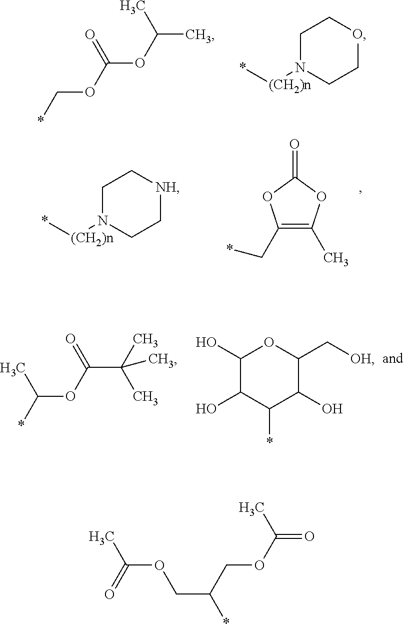
where n is 1, 2, 3, or 4 and * represents the point of attachment,
or a pharmaceutically acceptable salt thereof.

10. The compound of claim 1, represented by formula (VII): 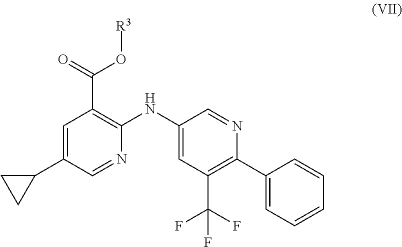
or a pharmaceutically acceptable salt thereof.

11. The compound of claim 10, wherein R³ is selected from the group consisting of: 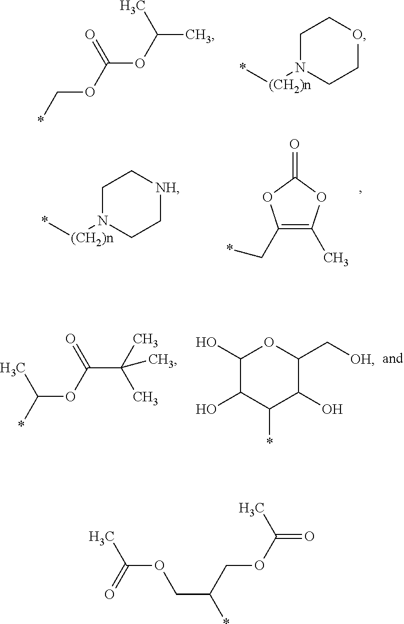
where n is 1, 2, 3, or 4 and * represents the point of attachment,
or a pharmaceutically acceptable salt thereof.

12. The compound of claim 1, represented by formula (VIII): 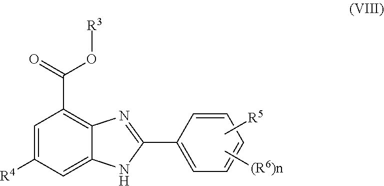
wherein R⁵ is an aryl or a heterocyclic aryl, wherein the aryl or the heterocyclic aryl is optionally substituted with one or more halogen and optionally substituted with one or more CF₃, and
R⁶ is a halogen or CF₃ and n=0, 1, 2, 3, or 4,
or a pharmaceutically acceptable salt thereof.

13. The compound of claim 12, wherein R³ is selected from the group consisting of: 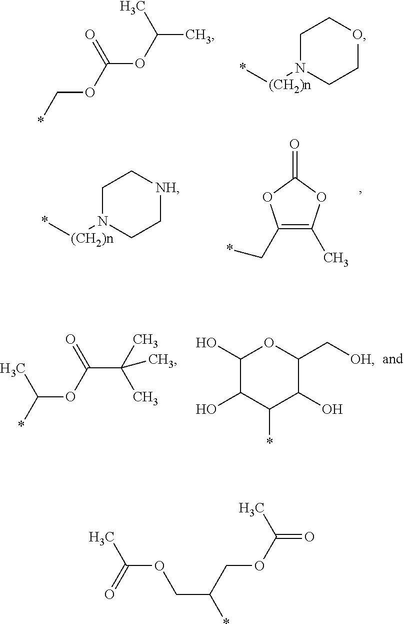
where n is 1, 2, 3, or 4 and * represents the point of attachment,
or a pharmaceutically acceptable salt thereof.

14. The compound of claim 1, represented by formula (IX): 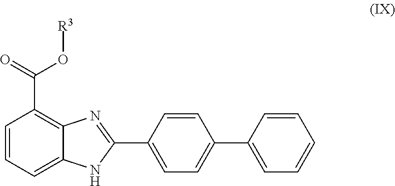
wherein the biphenyl is optionally substituted with one or two halogens,
or a pharmaceutically acceptable salt thereof.

15. The compound of claim 14, wherein R³ is selected from the group consisting of: 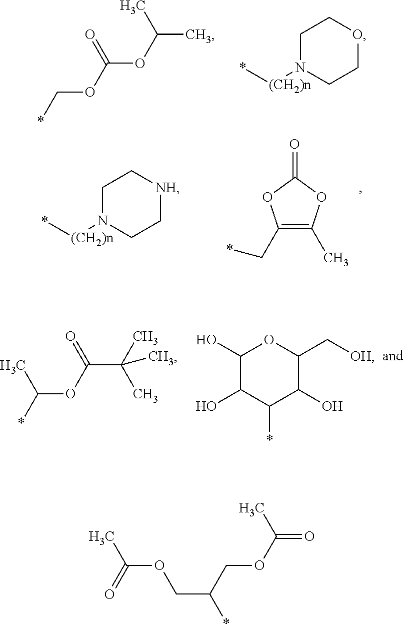
where n is 1, 2, 3, or 4 and * represents the point of attachment,
or a pharmaceutically acceptable salt thereof.

16. The compound of claim 1, wherein the compound is: 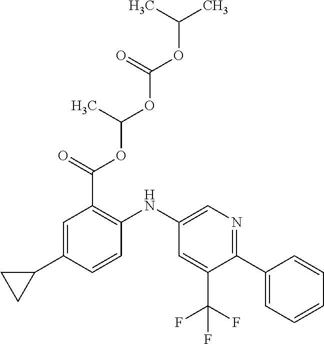 [SMILES: CC(C)OC(=O)OC(C)OC(=O)c1cc(C2CC2)ccc1Nc1cnc(-c2ccccc2)c(C(F)(F)F)c1]
or a pharmaceutically acceptable salt thereof.

17. The compound of claim 1, wherein the compound is: 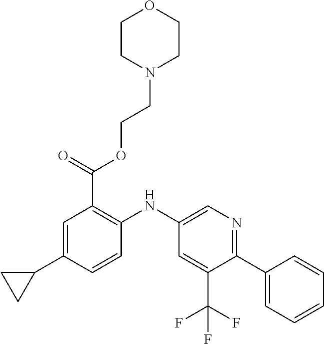 [SMILES: O=C(OCCN1CCOCC1)c1cc(C2CC2)ccc1Nc1cnc(-c2ccccc2)c(C(F)(F)F)c1]
or a pharmaceutically acceptable salt thereof.

18. The compound of claim 1, wherein the compound is: 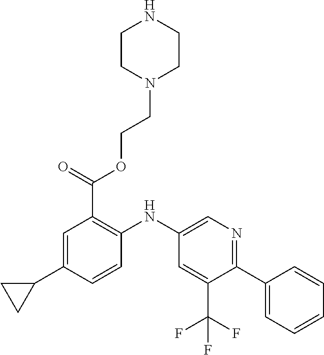 [SMILES: O=C(OCCN1CCNCC1)c1cc(C2CC2)ccc1Nc1cnc(-c2ccccc2)c(C(F)(F)F)c1]
or a pharmaceutically acceptable salt thereof.

19. The compound of claim 1, wherein the compound is: 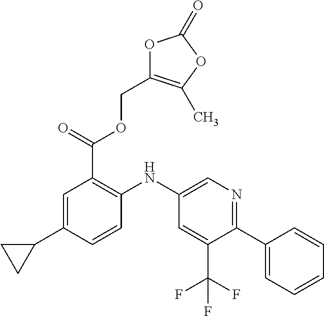 [SMILES: Cc1oc(=O)oc1COC(=O)c1cc(C2CC2)ccc1Nc1cnc(-c2ccccc2)c(C(F)(F)F)c1]
or a pharmaceutically acceptable salt thereof.

20. The compound of claim 1, wherein the compound is: 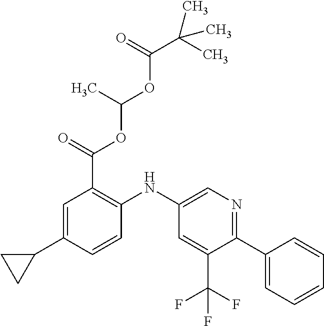 [SMILES: CC(OC(=O)c1cc(C2CC2)ccc1Nc1cnc(-c2ccccc2)c(C(F)(F)F)c1)OC(=O)C(C)(C)C]
or a pharmaceutically acceptable salt thereof.

21. The compound of claim 1, wherein the compound is: 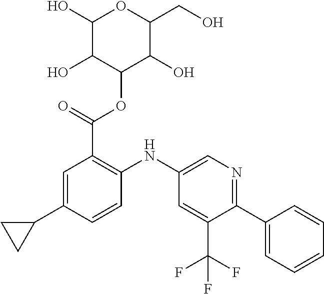 [SMILES: O=C(OC1C(O)C(O)OC(CO)C1O)c1cc(C2CC2)ccc1Nc1cnc(-c2ccccc2)c(C(F)(F)F)c1]
or a pharmaceutically acceptable salt thereof.

22. The compound of claim 1, wherein the compound is: 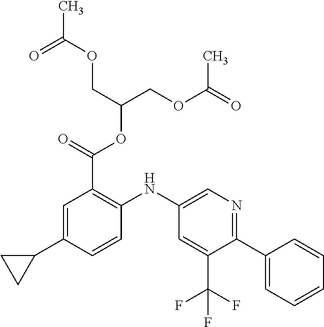 [SMILES: CC(=O)OCC(COC(C)=O)OC(=O)c1cc(C2CC2)ccc1Nc1cnc(-c2ccccc2)c(C(F)(F)F)c1]
or a pharmaceutically acceptable salt thereof.

23. A method of treating a condition or disease that is susceptible to amelioration by inhibition of DHODH, the method comprising administering to a subject a therapeutically effective does of the compound according to claim 1.
